(19) United States
(12) Patent Application Publication
(10) Pub. No.: US 2020/0105396 A1
(43) Pub. Date: Apr. 2, 2020

(54) PHOTOTHERAPEUTIC APPARATUS

(71) Applicants: Inforward, inc, Tokyo (JP); Public University Corporation Nagoya City University, Mizuho-ku (JP); Shibuya Corporation, Kanazawa-shi (JP)

(72) Inventors: Akimichi MORITA, Mizuho-ku (JP); Katsutoshi Sakano, Kanazawa-shi (JP)

(21) Appl. No.: 16/556,979

(22) Filed: Aug. 30, 2019

(30) Foreign Application Priority Data
Sep. 28, 2018 (JP) 2018-184964

(51) Int. Cl. G16H 20/40

(52) U.S. Cl. CPC G16H 20/40

(57) ABSTRACT

A phototherapeutic apparatus 1 includes: a doctor card 8B (external storage medium) into which permission data for permitting a change in an irradiation condition for light from the probe 3 is registered or a patient card 8A into which prohibition data for not permitting the change is registered; and a data reader 9 that reads the data of the external storage medium 8. When the data reader 9 reads the permission data from the doctor card 8B, the irradiation condition of the condition setter 7 is made changeable by using the monitor 10 (input part), and when the data reader 9 reads the prohibition data from the patient card 8A, the change in the irradiation condition of the condition setter 7 by using the monitor 10 is not permitted, and an irradiation condition registered in the patient card 8A together with the prohibition data is set into the condition setter 7.

### BACKGROUND OF THE INVENTION

### Field of the Invention

The present invention relates to a phototherapeutic apparatus. In more detail, the present invention relates to a phototherapeutic apparatus including a condition setter that sets an irradiation condition for light, and an input part that inputs the irradiation condition.

### Description of the Related Art

There is hitherto known a phototherapeutic apparatus that irradiates an affected part with light of ultraviolet A (UVA), ultraviolet B (UVB), or the like for the purpose of treating skin diseases such as psoriasis, vitiligo, or atopic dermatitis.

In the phototherapeutic apparatus as above, irradiation conditions, such as an irradiation output and irradiation time, for light to be applied are different for each affected part. Hence in the phototherapeutic apparatus, a condition setter is provided to register the irradiation conditions, and an input part is also provided to register the irradiation conditions into the condition setter.

As one obtained by making such a phototherapeutic apparatus portable, there is known a phototherapeutic apparatus in which a photo-irradiation unit for performing irradiation with light is provided in a probe that can be held by a user (Japanese Patent Laid-Open No. 2017-164417).

In treatment with light by using such a phototherapeutic apparatus, if the phototherapeutic apparatus is used by a doctor in a medical institution while the same phototherapeutic apparatus is also used at the home of the patient for the home treatment, it is possible to share the phototherapeutic apparatus, which is effective.

However, in that case, it is necessary to facilitate the doctor to set irradiation conditions for light in the medical institution, while making the irradiation conditions unchangeable by the patient in the home treatment so as to prevent the occurrence of an accident due to irradiation with excess light or the like caused by the patient operating the apparatus to change the irradiation conditions for light.

In view of such a problem, an object of the present invention is to provide a phototherapeutic apparatus capable of preventing an irradiation condition from being changed by a patient when the apparatus is used for home treatment, and capable of facilitating the setting of the irradiation condition when the doctor performs treatment.

### SUMMARY OF THE INVENTION

That is, a phototherapeutic apparatus according to the invention of claim 1 is a phototherapeutic apparatus including: a probe held and operated by a user; a photo-irradiation unit that is provided in the probe and irradiates an affected part with light; an irradiation controller that controls the light of the photo-irradiation unit; a condition setter that sets an irradiation condition for the light by the irradiation controller; and an input part, with which the irradiation condition is input. The apparatus includes an external storage medium, into which one of permission data and prohibition data is registered, the permission data permitting a change in the irradiation condition of the condition setter, the prohibition data not permitting the change, and a data reader that reads the data of the external storage medium. When the data reader reads the permission data registered in the external storage medium, the irradiation condition of the condition setter is made changeable by using the input part. When the data reader reads the prohibition data registered in the external storage medium, the change in the irradiation condition of the condition setter by using the input part is not permitted, and an irradiation condition registered in the external storage medium together with the prohibition data is set into the condition setter.

According to the invention of claim 1 above, when the permission Liata registered in the external storage medium is read, a doctor being the user can change the irradiation condition of the condition setter by using the input part, and the doctor can use the phototherapeutic apparatus according to the present invention to treat the patient in the medical institution.

On the other hand, when the prohibition data registered in the external storage medium is read, a patient being the user cannot change the irradiation condition of the condition setter by using the input part, but instead, the irradiation condition registered in the external storage medium is registered into the condition setter. Therefore, the affected part of the patient can be irradiated with appropriate light, and the patient can safely use the phototherapeutic apparatus according to the present invention for the home treatment.

### BRIEF DESCRIPTION OF THE DRAWING

FIG. 1 is a configuration view of a phototherapeutic apparatus according to the present embodiment.

### DETAILED DESCRIPTION OF THE PREFERRED EMBODIMENT

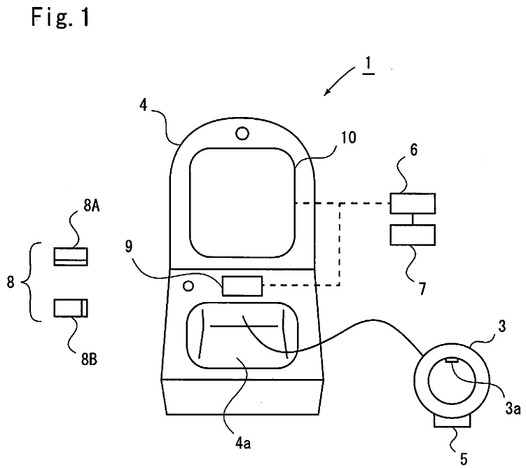

In the following, the illustrated embodiment will be described. FIG. 1 illustrates a phototherapeutic apparatus 1, and the phototherapeutic apparatus 1 is an apparatus for treating skin diseases such as psoriasis, vitiligo, and atopic dermatitis.

The phototherapeutic apparatus 1 of the present embodiment can be used both by the medical doctor for treatment of a patient in a medical institution such as a hospital, and by the patient for home treatment that is performed in the home of the patient, and the like.

The phototherapeutic apparatus 1 includes a probe 3 that can be held by the user, and a body 4 connected to the probe 3 through a cable. The body 4 is formed with a container 4a capable of keeping the probe 3, and the user can keep the probe 3 and other auxiliaries in the container 4a to easily carry the phototherapeutic apparatus 1. Note that the connection between the probe 3 and the body 4 is not necessarily wired, but may be of a wireless system.

In the following description, the patient is also a user to operate the phototherapeutic apparatus 1. However, a person other than the patient, such as a nursing attendant who tends the patient, can also operate the apparatus, and when the phototherapeutic apparatus 1 is used in the medical institution, the doctor is the user.

The probe 3 is provided with a photo-irradiation unit 5 that irradiates the skin of the patient with a so-called narrow-band UVB light at a wavelength of around 311 nm. Irradiating the affected part of the patient with the light at the above wavelength enables the treatment of the skin disease as described above.

The probe 3 includes a trigger switch 3a having a shape easy for the user to operate and configured to instruct the start of irradiation with the light from the photo-irradiation unit 5. Further, the probe 3 includes a cover (not illustrated) to prevent the incidence of the light, applied from the photo-irradiation unit 5, directly on the eye of the patient.

In the probe 3 of the present embodiment, operation switches, except for the trigger switch 3a, and the like are omitted to prevent settings of irradiation conditions for the light from the probe 3, thereby preventing the patient from performing unnecessary operation or erroneous operation.

The body 4 includes: an irradiation controller 6 that controls the light applied from the photo-irradiation unit 5 based on the operation of the probe 3; a condition setter 7, into which the irradiation conditions for the light are set by the irradiation controller 6; a data reader 9 that reads an external storage medium 8 carried by the doctor or the patient; and a monitor 10 that shows the doctor a setting screen for the irradiation conditions for the light, while showing the patient an operation method for the probe 3.

The irradiation controller 6 controls an irradiation output, irradiation time, and irradiation frequency for light applied from the photo-irradiation unit 5. The irradiation conditions such as the irradiation output, irradiation time, and irradiation frequency are registered into the condition setter 7.

When the trigger switch 3a of the probe 3 is operated, the irradiation controller 6 causes the photo-irradiation unit 5 of the probe 3 to perform irradiation with the light in accordance with the irradiation conditions of the condition setter 7. Note that an irradiation amount, obtained by multiplying the irradiation output by the irradiation time, can also be set as the irradiation condition.

As the external storage medium 8, a conventionally known IC card can be used, and the data reader 9 which reads the external storage medium 8 is provided with a conventionally known IC card reader.

The external storage medium 8 includes two kinds of cards which are a patient card 8A carried by the patient and a doctor card 8B carried by the doctor. By causing the data reader 9 to read either the patient card 8A or the doctor card 8B, the phototherapeutic apparatus 1 can be used for the home treatment or the treatment by the doctor.

When the power supply of the phototherapeutic apparatus 1 is turned on, the monitor 10 displays an instruction to urge the insertion of the external storage medium 8 into the data reader 9. Thereafter, when the patient card 8A or the doctor card 8B is inserted, the monitor 10 displays an instruction to input a password previously registered in each card.

Further, the data reader 9 stores a time at which the patient card 8A is inserted. In a case where the treatment is performed using the same patient card 8A, when, for example, 18 hours has not elapsed from the last treatment, the monitor 10 displays that the treatment cannot be performed for the sake of the safety.

In the patient card 8A, there are registered prohibition data for not permitting a change in the irradiation condition of the condition setter 7, irradiation conditions previously registered for each affected part by the doctor, and operation instruction data for instructing the patient to operate the probe 3.

When the patient card 8A in which the prohibition data is registered is read by the data reader 9, the setting screen for setting the irradiation conditions for the light is not displayed on the monitor 10, so that the patient cannot freely change the irradiation conditions.

Meanwhile, in the patient card 8A, the irradiation conditions such as the irradiation output, irradiation time, and irradiation frequency for the light are registered for each affected part based on the diagnosis by the doctor. When the data reader 9 reads the patient card 8A, the irradiation conditions are registered into the condition setter 7.

While the irradiation conditions can be registered into the patient card 8A by using a personal computer or the like provided in the medical institution via an IC card writer or the like, it is also possible to provide the data reader 9 of the phototherapeutic apparatus 1 with the function of writing the irradiation conditions.

In that case, first, the data reader 9 may be caused to read the doctor card 8B, and the doctor may then set the irradiation conditions for the light beam. Thereafter, the patient card 8A may be inserted into the data reader 9, and the set irradiation conditions may be written into the patient card 8A.

As the operation instruction data registered in the patient card 8A, an actual picture or video of the affected part, or animation or illustration instead of the actual picture or video, is registered, and this is displayed on the monitor 10, thereby indicating the position to be touched with the probe 3 by the patient.

Note that a region to be treated can also be presented by a ,voice or the like together with the video or the like displayed on the monitor 10 by registering data of a voice or the like as the operation instruction data and further providing a voice output part in the body 4.

In the doctor card 8B, there is registered permission data for permitting a change in the irradiation conditions of the condition setter 7.

When the doctor card 8B in which the permission data is registered is read by the data reader 9, the setting screen for setting the irradiation conditions for the light is displayed on the monitor 10, and the doctor can change the irradiation conditions for the light to be applied while diagnosing the affected part of the patient.

The doctor card 8B of the present embodiment can be used by the doctor as a key for treating the patient by using the phototherapeutic apparatus 1. In particular, the doctor card 8B is prevented from being used by anyone but the doctor by asking for the input of the password when the doctor card 8B is read by the data reader 9.

The monitor 10 is a conventionally known touch-panel monitor and is used as an operation instruction unit to instruct the patient to operate the probe 3, or as an input part for the doctor to set the irradiation conditions for the light. The monitor 10 is also used at the time of inputting the password of each card read into the data reader 9 after the power supply is turned on.

As described above, when the data reader 9 reads the patient card 8A, the monitor 10 displays the operation instruction data registered in the patient card 8A, whereby the patient can accurately position the probe 3 on the affected part while viewing the image or the like displayed on the monitor 10.

When the user operates the trigger switch 3a of the probe 3 in that state, the irradiation controller 6 causes the photo-irradiation unit 5 to perform irradiation with the light in accordance with the irradiation conditions set in the condition setter 7 via the patient card 8A, thereby enabling appropriate treatment of the affected part.

On the other hand, when the data reader 9 reads the doctor card 8B, the monitor 10 displays the setting screen for inputting the irradiation conditions for the light beam, so that the doctor can input an appropriate irradiation output, irradiation time, and the like for each affected part, while touching the monitor 10.

Then, the doctor holds and moves the probe 3 to the affected part of the patient and then operates the trigger switch 3a. Therefore, in accordance with the irradiation conditions set by the doctor, the irradiation controller 6 causes the photo-irradiation unit 5 to irradiate the affected part with the light with the irradiation output and the irradiation time suitable for the affected part, thereby enabling appropriate treatment of the affected part with arbitrarily irradiation frequency.

According to the phototherapeutic apparatus 1 having the above configuration, the phototherapeutic apparatus 1 can be used both by the doctor in the medical institution and by the patient for home treatment at home, thereby eliminating the need to separately manufacture the phototherapeutic apparatus 1 for the medical institution and the phototherapeutic apparatus 1 for the home treatment.

Moreover, the phototherapeutic apparatus 1 of the present embodiment permits the doctor to change the irradiation conditions while prohibiting the patient from changing the irradiation condition by causing the data reader 9 to read the doctor card 8B which is carried by the doctor and has the permission data registered therein, or the patient card 8A which is carried by the patient and has the prohibition data registered therein.

That is, when the doctor card 8B is used, similarly to the conventionally known phototherapeutic apparatus 1, in the medical institution, the doctor can freely set the irradiation conditions for the light to be applied, and can set appropriate irradiation conditions with respect to the affected part, to perform appropriate treatment using the probe 3.

On the other hand, when the patient card 8A is used, the patient cannot change the irradiation conditions for the light beam by himself or herself, so that no operation error occurs due to an erroneous setting, and it is possible to perform proper home treatment based on the doctor's instruction in accordance with the irradiation conditions registered in the patient card 8A.

The light to be applied by the phototherapeutic apparatus according to the present invention is not limited to the above-mentioned narrow-band UVB for treating skin diseases, but laser beams for removing a bruise, a macula, a mole, and the like can also be considered. The configuration of the present embodiment is applicable to anything requiring the settings of irradiation conditions for such light as above so as to perform treatment using the light.

### REFERENCE SIGNS LIST

1 phototherapeutic apparatus
3 probe
4 body
5 photo-irradiation unit
6 irradiation controller
7 condition setter
external storage medium
8A patient card
8B doctor card
9 data reader
10 monitor (input part)

What is claimed is:

1. A phototherapeutic apparatus comprising:
a probe held and operated by a user;
a photo-irradiation unit that is provided in the probe and irradiates an affected part with light;
an irradiation controller that controls the light of the photo-irradiation unit;
a condition setter that sets an irradiation condition for the light by the irradiation controller; and
an input part, with which the irradiation condition is input,
characterized in that
the apparatus comprises
an external storage medium, into which one of permission data and prohibition data is registered, the permission data permitting a change in the irradiation condition of the condition setter, the prohibition data not permitting the change, and
a data reader that reads the data of the external storage medium,
when the data reader reads the permission data registered in the external storage medium, the irradiation condition of the condition setter is made changeable by using the input part, and
when the data reader reads the prohibition data registered in the external storage medium, the change in the irradiation condition of the condition setter by using the input part is not permitted, and an irradiation condition registered in the external storage medium together with the prohibition data is set into the condition setter.

2. The phototherapeutic apparatus according to claim 1, characterized in that
the input part is made of a touch panel that displays a necessary screen and is touched so that input operation can be performed, and
when the data reader reads the permission data from the external storage medium, a screen for setting the irradiation condition is displayed on the input part to enable the irradiation condition to be input.
